(19) United States
(12) Patent Application Publication
(10) Pub. No.: US 2011/0111392 A1
(43) Pub. Date: May 12, 2011

(54) INTEGRATED ENHANCED CHEMILUMINESCENCE BIOSENSORS

(75) Inventors: Tao Pan, Shanghhai (CN); Zhenhong Sun, Shanghai (CN); Wendy Wang, Shanghai (CN); Xuanbin Liu, Shanghai (CN)

(73) Assignee: HONEYWELL INTERNATIONAL INC., Morristown, NJ (US)

(21) Appl. No.: 12/988,797

(22) PCT Filed: Apr. 21, 2008
(86) PCT No.: PCT/CN08/00810

§ 371 Date: Dec. 23, 2010

(51) Int. Cl. C12Q 1/70; G01N 21/00; C12Q 1/00; C12Q 1/66; C12Q 1/28; C12Q 1/02; C12M 1/34; G01N 33/00; G01N 30/02; G01N 27/447

(52) U.S. Cl. 435/5; 422/82.05; 435/4; 435/8; 435/28; 435/29; 435/287.3; 436/71; 436/73; 436/86; 436/94; 436/161; 204/451; 204/452

(57) ABSTRACT

A method and apparatus for determining the concentration of an analyte in a sample is provided. This method involves combining enhanced chemiluminescence with microchip capillary electrophoresis or microchip liquid chromatography.

### BACKGROUND OF THE INVENTION

Microscale separations such as capillary liquid chromatography (LC) and capillary electrophoresis (CE) offer shorter analysis times, low reagent and solvent consumption, increased reliability and high performance over traditional separations. The use of microfluidic devices to perform these types of separations provides advantages in instrumental integration and portability. The increasing popularity of capillary liquid chromatography and capillary electrophoresis over the last 25 years, and the more recent transition to microfluidic devices in the last 15 years, has created a need for detection systems that are amenable to miniaturization. Due to the low flow rates (tens of nanoliters/min to tens of μL/min) and very small volumes used in capillary liquid chromatography and capillary electrophoresis (tens of nL), these systems must provide very high mass sensitivity (picomol or less) and chemical selectivity, and have the ability to measure analytes of interest in intended applications without prior chemical derivatization. Additionally, detectors should be easy to use, possess high stability and reproducibility, and be easily fabricated in appropriate dimensions at a reasonable cost.

Chemiluminescence detection is very mass sensitive. Many analytes, including many pharmaceutical drugs and endogenous neurotransmitters or neuroactive compounds, are able to give light through chemical and physical reactions, which allow them to be measured by luminescence detection. Chemiluminescence detection scales very well with reduced sample volume, making it amenable to miniaturization.

What is needed is a quantitative chemiluminescence detection method and apparatus that offers the robustness required for use in an analytical laboratory.

### BRIEF DESCRIPTION OF THE DRAWINGS

Embodiments of the invention may be best understood by referring to the following description and accompanying drawings, which illustrate such embodiments. In the drawings:

FIG. 1 illustrates a cross-sectional view of an exemplary sensor device for determining the concentration of an analyte in a sample.

FIG. 2 illustrates a cross-sectional view of an exemplary sensor device for determining the concentration of an analyte in a sample.

### DETAILED DESCRIPTION

The present invention provides a method and an apparatus or sensor for determining the concentration of an analyte in a sample by combining enhanced chemiluminescence with microchip capillary electrophoresis or microchip liquid chromatography. This combines the advantages of enhanced chemiluminescence (e.g., ultrasensitive, low cost, and versatile) and the advantages of microchip capillary electrophoresis or microchip liquid chromatography (e.g., rapid throughput, simple design, and high sensitivity). As a result, this apparatus is much smaller than any currently used.

As used herein, certain terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 11^{th} Edition, by Sax and Lewis, Van Nostrand Reinhold, New York, N.Y., 1987, and The Merck Index, 11^{th} Edition, Merck & Co., Rahway N.J. 1989.

As used herein, the term “and/or” means any one of the items, any combination of the items, or all of the items with which this term is associated.

As used herein, the singular forms “a,” “an,” and “the” may include plural reference unless the context clearly dictates otherwise. Therefore, for example, a reference to “a formulation” may include a plurality of such formulations, so that a formulation of compound X may include formulations of compound X.

As used herein, the term “about” means a variation of 10 percent of the value specified, for example, about 50 percent carries a variation from 45 to 55 percent. For integer ranges, the term about can include one or two integers greater than and less than a recited integer.

As used herein, the term “analyte” refers to a substance to be detected, which is included in the samples being separated using microchip capillary electrophoresis. For example, analytes can include antigenic substances, haptens, antibody, toxins, inorganic species (e.g., metal ions, metals, NO₃⁻, Cl⁻, etc.) organic compounds, proteins, peptides, microorganisms, amino acids, nucleic acids, hormones, steroids, vitamins, drugs (including those administered for therapeutic purposes as well as those administered for illicit purposes), bacteria, virus particles, metabolites of or antibodies to any of the above substances.

As used herein, the term “charge-coupled device” refers to a device for forming images electronically, using a layer of silicon that releases electrons when struck by incoming light.

As used herein, the term “enhanced chemiluminescence” refers to the combination of any luminescences (e.g., commonly used “chemiluminescence,” “electrochemical luminescence,” or “bioluminescence” etc.) that are triggered by chemicals (e.g., chemical reaction, attachment of chemiluminescence active tag and the like), physical reactions (e.g., photons, electrons/potentials) and/or electrochemical reaction (e.g., redox reaction).

As used herein, the term “liquid” refers to a substance that undergoes continuous deformation under a shearing stress. See, e.g., Concise Chemical and Technical Dictionary, 4^{th} Edition, Chemical Publishing Co., Inc., p. 707, New York, N.Y. (1986).

As used herein, the phrase “in one embodiment” refers a particular feature, structure, or characteristic. However, every embodiment may not necessarily include the particular feature, structure, or characteristic. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the term “microchip capillary electrophoresis” refers to the capillary electrophoresis that is carried out using a microchip based capillary electrophoresis device.

As used herein, the term “liquid chromatography” refers to chromatography in which the mobile phase is a liquid.

As used herein, the term “sample” refers to a material suspected of containing the analyte. The sample can be used directly as obtained from the source or following a pretreatment to modify the character of the sample. The sample can be derived from any industrial (e.g., food industry, pharmaceutical, etc.), agricultural; environmental (e.g., water, soil, etc), and biological source, such as a physiological fluid, including, blood, saliva, ocular lens fluid, cerebral spinal fluid, sweat, urine, milk, ascites fluid, raucous, synovial fluid, peritoneal fluid, amniotic fluid or the like. The sample can be pretreated prior to use, such as preparing plasma from blood, diluting viscous fluids, and the like. Methods of treatment can involve filtration, distillation, concentration, inactivation of interfering components, and the addition of reagents. Besides physiological fluids, other liquid samples can be used such as water, food products, and the like for the performance of environmental or food production assays. In addition, a solid material suspected of containing the analyte can be used as the sample. In some instances it may be beneficial to modify a solid test sample to form a liquid medium or to release the analyte.

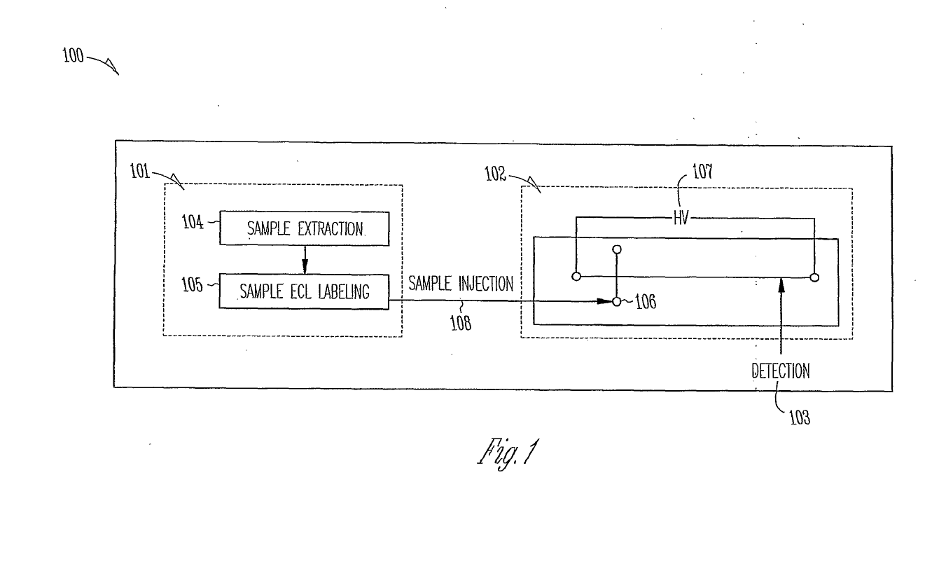

FIG. 1 is a cross-sectional view of an exemplary sensor device (100) for determining the concentration of an analyte in a sample. The sensor device (100) includes an enhanced chemiluminescence preparation device (101); a microchip capillary electrophoresis device (102); and a detector (103). The enhanced chemiluminescence preparation device (101) includes a sample extraction device (104) and a sample labeling device (105). The microchip capillary electrophoresis device (102) includes a sample injection port (106) and a high voltage potential (107). The enhanced chemiluminescence preparation device (101) and the microchip chemiluminescence device (102) are physically connected by the sample injection tube (108).

In this method, a sample may contain an analyte. The sample, for example, is introduced into the enhanced chemiluminescence preparation device (101) in which a specific enhanced chemiluminescence label is attached to the analyte.

The sample containing the analyte is injected into the microchip capillary electrophoresis device (102), a high voltage potential is applied to separate the analyte from other materials in the sample on the basis of charge, size, or both.

The enhanced chemiluminescence response of the analyte is detected by a detector (103) and the response compared with a calibration curve to provide the concentration of the analyte in the sample. Likewise, this process may be repeated using other enhanced chemiluminescence labels that are specific for other analytes. As a result, the concentrations of several analytes may be determined. In one embodiment, several analytes may have the same chemiluminescence label. In another embodiment, several analytes may have different chemiluminescence labels.

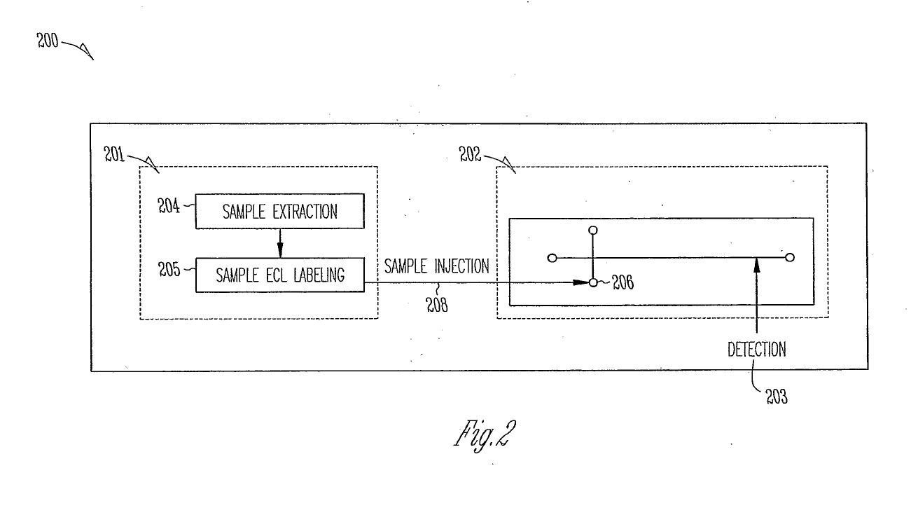

FIG. 2 is a cross-sectional view of an exemplary sensor device (200) for determining the concentration of an analyte in a sample. The sensor device (200) includes an enhanced chemiluminescence preparation device (201); a microchip liquid chromatography device (202); and a detector (203). The enhanced chemiluminescence preparation device (201) includes a sample extraction device (204) and a sample labeling device (205). The microchip liquid chromatography device (202) includes a sample injection port (206). The enhanced chemiluminescence preparation device (201) and the microchip liquid chromatography device (202) are physically connected by the sample injection tube (208).

In this method, a sample may contain an analyte. The sample, for example, is introduced into the enhanced chemiluminescence preparation device (201) in which a specific enhanced chemiluminescence label is attached to the analyte.

The sample containing the analyte is injected into the microchip liquid chromatography device (202); a pressure flow is applied to separate the analyte from other materials in the sample on the basis of charge, size, or both.

The enhanced chemiluminescence response of the analyte is detected by a detector (203) and the response compared with a calibration curve to provide the concentration of the analyte in the sample. Likewise, this process may be repeated using other enhanced chemiluminescence labels that are specific for other analytes. As a result, the concentrations of several analytes may be determined. In one embodiment, several analytes may have the same chemiluminescence label. In another embodiment, several analytes may have different chemiluminescence labels.

In one embodiment, a method for determining the concentration of an analyte in a sample is provided. The method includes labeling an analyte with an enhanced chemiluminescence label to provide an enhanced chemiluminescence labeled analyte; introducing the enhanced chemiluminescence labeled analyte into a microchip capillary electrophoresis device; applying an electric field across the microchip capillary electrophoresis device to separate the enhanced chemiluminescence labeled analyte from another material in the sample; detecting an enhanced chemiluminescence response from the enhanced chemiluminescence labeled analyte; and comparing the enhanced chemiluminescence response with a calibration curve to determine the concentration of the analyte in the sample.

In one embodiment, a method for determining the concentration of an analyte in a sample is provided. The method includes labeling an analyte with an enhanced chemiluminescence label to provide an enhanced chemiluminescence labeled analyte; introducing the enhanced chemiluminescence labeled analyte into a microchip liquid chromatography device; applying a pressure flow to the microchip liquid chromatography device to separate the enhanced chemiluminescence labeled analyte from another material in the sample; detecting an enhanced chemiluminescence response from the enhanced chemiluminescence labeled analyte; and comparing the enhanced chemiluminescence response with a calibration curve to determine the concentration of the analyte in the sample.

In one embodiment, the analyte includes an antibody, an antibiotic, an antigen, a bacterium, a carbohydrate, a cell, a drug, an enzyme, a hormone, a lectin, a herbicide, a lipid, an ion, a metal, a pesticide, a protein, a peptide, a nucleic acid molecule, a spore, a toxin, a virus, a metal oxide, a silicon oxide, a phosphate, a nanoparticle, or a combination thereof.

In one embodiment, the enhanced chemiluminescence label includes an acridinium compound, luminol, luciferase, horseradish peroxidase, β-galactosidase fluorescein isothiocyanate, Ru(bipy)₃²⁺, pyrogallol, quantum dots, or a combination thereof.

In one embodiment, the another material includes another analyte.

In one embodiment, the detecting of the response is performed with a charge-coupled device, a camera, a video camera, a silicon photo-cell, a photomultiplier tube, or a combination thereof.

In one embodiment, a sensor device is provided. The sensor includes an enhanced chemiluminescence preparation device to combine an enhanced chemiluminescence label to an analyte in a sample; a microchip device coupled to the enhanced chemiluminescence preparation device to receive a sample from the enhanced chemiluminescence preparation device and to separate the analyte from another material in the sample; a detector coupled to the microchip device to detect an enhanced chemiluminescence response from the enhanced chemiluminescence label combined with the analyte in the sample; and an analyzer coupled to the detector to determine the concentration of an analyte in a sample.

In one embodiment, the microchip device includes a microchip capillary electrophoresis device or a microchip liquid chromatography device.

In one embodiment, the microchip device is a microchip capillary electrophoresis device. In one embodiment, the microchip device is a microchip liquid chromatography device.

Any suitable enhanced chemiluminescence preparation device may be used and are well known in the art. Enhanced chemiluminescence is a common technique in biology. For example, a horseradish peroxidase enzyme is tethered to the molecule of interest, typically through labeling an immunoglobulin that specifically recognizes the molecule. This enzyme complex, catalyzes the conversion of the enhanced chemiluminescence substrate into a sensitized reagent in the vicinity of the molecule of interest, which on further oxidation by hydrogen peroxide, produces a triplet carbonyl, which emits light when it decays to the singlet carbonyl. Enhanced chemiluminescence allows detection of minute quantities of a biomolecule. Proteins can be detected down to femtomole quantities, well below the detection limit for most assay systems.

Any suitable microchip capillary electrophoresis device may be used including for example, the commercially available systems that are used for routine analysis.

Any suitable microchip liquid chromatography device may be used including, for example, the device described in U.S. Pat. No. 6,342,142.

The sensor for determining the concentration of analyte in a sample may be operated at, for example, any desired temperature, pressure, and flow rate that may be suitable for the analyte.

In an exemplary embodiment, the sensor may be operated at a temperature from about 0° C. to about 100° C., typically from about 10° C. to about 60° C., and more typically about room temperature to about 30° C.

The following examples are intended to illustrate the above invention and should not be construed as to narrow its scope. One skilled in the art will readily recognize that the examples suggest many other ways in which the invention could be practiced. It should be understood that numerous variations and modifications may be made while remaining within the scope of the invention.

### EXAMPLES

### Example 1

A sample that contains various analytes is mixed together with antibodies that contain a horseradish peroxidase label in the sample preparation device. The mixture is pumped into the microchip capillary electrophoresis device, a high voltage is applied, and the labeled analytes are detected by the detector.

### Example 2

A sample that contains various analytes is mixed together with antibodies that contain a Ru(bipy)₃²⁺ labels in the sample preparation device. The mixture is pumped into the microchip capillary electrophoresis device, a high voltage is applied, and the labeled analytes are detected by the detector.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicant reserves the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

What is claimed is:

1. A method for determining the concentration of an analyte in a sample comprising:
labeling an analyte with an enhanced chemiluminescence label to provide an enhanced chemiluminescence labeled analyte;
introducing the enhanced chemiluminescence labeled analyte into a microchip capillary electrophoresis device;
applying an electric field across the microchip capillary electrophoresis device to separate the enhanced chemiluminescence labeled analyte from another material in the sample;
detecting an enhanced chemiluminescence response from the enhanced chemiluminescence labeled analyte; and
comparing the enhanced chemiluminescence response with a calibration curve to determine the concentration of the analyte in the sample.

2. The method of claim 1, wherein the analyte comprises an antibody, an antibiotic, an antigen, a bacterium, a carbohydrate, a cell, a drug, an enzyme, a hormone, a lectin, a herbicide, a lipid, an ion, a metal, a pesticide, a protein, a peptide, a nucleic acid molecule, a spore, a toxin, a virus, a metal oxide, a silicon oxide, a phosphate, a nanoparticle, or a combination thereof.

3. The method of claim 1, wherein the enhanced chemiluminescence label comprises an acridinium compound, luminol, luciferase, horseradish peroxidase, β-galactosidase fluorescein isothiocyanate, Ru(bipy)₃²⁺, pyrogallol, quantum dots, or a combination thereof.

4. The method of claim 1, wherein the another material comprises another analyte.

5. The method of claim 1, wherein the detecting of the response is performed with a charge-coupled device, a camera, a video camera, a silicon photo-cell, a photomultiplier tube, or a combination thereof.

6. A method for determining the concentration of an analyte in a sample comprising:
labeling an analyte with an enhanced chemiluminescence label to provide an enhanced chemiluminescence labeled analyte;
introducing the enhanced chemiluminescence labeled analyte into a microchip liquid chromatography device;
applying a pressure flow to the microchip liquid chromatography device to separate the enhanced chemiluminescence labeled analyte from another material in the sample;
detecting an enhanced chemiluminescence response from the enhanced chemiluminescence labeled analyte; and
comparing the enhanced chemiluminescence response with a calibration curve to determine the concentration of the analyte in the sample.

7. The method of claim 6, wherein the analyte comprises an antibody, an antibiotic, an antigen, a bacterium, a carbohydrate, a cell, a drug, an enzyme, a hormone, a lectin, a herbicide, a lipid, an ion, a metal, a pesticide, a protein, a peptide, a nucleic acid molecule, a spore, a toxin, a virus, a metal oxide, a silicon oxide, a phosphate, a nanoparticle, or a combination thereof.

8. The method of claim 6, wherein the enhanced chemiluminescence label comprises an acridinium compound, luminol, luciferase, horseradish peroxidase, β-galactosidase fluorescein isothiocyanate, Ru(bipy)₃²⁺, pyrogallol, quantum dots, or a combination thereof.

9. The method of claim 6, wherein the another material comprises another analyte.

10. The method of claim 6, wherein the detecting of the response is performed with a charge-coupled device, a camera, a video camera, a silicon photo-cell, a photomultiplier tube, or a combination thereof.

11. A sensor device, comprising:
an enhanced chemiluminescence preparation device to combine an enhanced chemiluminescence label to an analyte in a sample;
a microchip device coupled to the enhanced chemiluminescence preparation device to receive a sample from the enhanced chemiluminescence preparation device and to separate the analyte from another material in the sample;
a detector coupled to the microchip device to detect an enhanced chemiluminescence response from the enhanced chemiluminescence label combined with the analyte in the sample; and
an analyzer coupled to the detector to determine the concentration of the analyte in a sample.

12. The sensor device of claim 11, wherein the microchip device comprises a microchip capillary electrophoresis device or a microchip liquid chromatography device.

13. The sensor device of claim 12, wherein the microchip device is a microchip capillary electrophoresis device.

14. The sensor device of claim 12, wherein the microchip device is a microchip liquid chromatography device.

15. The sensor device of claim 11, wherein the detector comprises a charge-coupled device, a camera, a video camera, a photo-cell, a photomultiplier tube, or a combination thereof.

16. The sensor device of claim 11, wherein the analyte comprises an antibody, an antibiotic, an antigen, a bacterium, a carbohydrate, a cell, a drug, an enzyme, a hormone, a lectin, a herbicide, a lipid, an ion, a metal, a pesticide, a protein, a peptide, a nucleic acid molecule, a spore, a toxin, a virus, a metal oxide, a silicon oxide, a phosphate, a nanoparticle, or a combination thereof.

17. The sensor device of claim 11, wherein the enhanced chemiluminescence label comprises an acridinium compound, luminol, luciferase, horseradish peroxidase, β-galactosidase fluorescein isothiocyanate, Ru(bipy)₃²⁺, pyrogallol, quantum dots, or a combination thereof.

18. The sensor device of claim 11, wherein the another material comprises another analyte.
